(19) United States
(12) Patent Application Publication
(10) Pub. No.: US 2024/0026273 A1
(43) Pub. Date: Jan. 25, 2024

(54) CELL CULTURE SYSTEM, METHOD AND ASSEMBLY

(71) Applicants: UNIVERSITÉ D’AIX MARSEILLE, MARSEILLE (FR); CENTRE NATIONAL DE LA RECHERCHE SCIENTIFIQUE, PARIS (FR); CENTRALE MARSEILLE, MARSEILLE (FR)

(72) Inventors: Olivier DETOURNAY, MARSEILLE (FR); Patrice MEUNIER, MARSEILLE (FR)

(21) Appl. No.: 18/266,242

(22) PCT Filed: Dec. 10, 2021
(86) PCT No.: PCT/EP2021/085241

§ 371 Date: Jun. 8, 2023

(30) Foreign Application Priority Data
Dec. 11, 2020 (FR) 2013039

(51) Int. Cl. C12M 1/00; C12M 3/04; C12M 1/12; C12M 1/107

(52) U.S. Cl. CPC C12M 29/04; C12M 27/10; C12M 23/06; C12M 21/02; C12M 23/22; C12M 23/36

(57) ABSTRACT

A cell culture system includes a first component having an opening at the bottom, a membrane for diffusing a gas into a liquid by dissolution, an enclosure able to hold a volume of gas that can diffuse into the liquid via the membrane and a drive member for driving the container, the container being inclined at a non-zero angle α that is less than or equal to 30°. A cell culture system comprising a first component and a second component forming a ring-shaped container and a drive member, the container being inclined at a non-zero angle α that is less than or equal to 30°. A method using a system of the invention and to an assembly comprising a system of the invention and a liquid containing a species that is to be cultured.

### CROSS-REFERENCE TO RELATED APPLICATIONS

This application is a National Stage of International patent application PCT/EP2021/085241, filed on Dec. 10, 2021, which claims priority to foreign French patent application No. FR 2013039, filed on Dec. 11, 2020, the disclosures of which are incorporated by reference in their entirety.

### FIELD OF THE INVENTION

The present invention relates to a cell culture system and to a cell culture method using such a system. The present invention relates in particular to a cell culture system comprising a first component open at the top, a membrane for diffusing a gas into a liquid by dissolution, the membrane and the first component forming a container, an enclosure able to hold a volume of gas, a drive member for driving the container, the container being inclined at an angle α that is non-zero and less than 30° with respect to the vertical direction. The present invention also relates to a cell culture method using a system as described hereinabove, the method comprising a step of supplying a liquid containing a species that is to be cultured and a rotating of the container by the drive member.

### BACKGROUND

Cell culture is facing a significant economical challenge regarding the large-scale production of fragile cells. Certain cells may be used directly for cell therapy and others may be used for producing high added value molecules of interest. Specifically, certain species are capable of producing molecules of interest naturally or following modification of the species to produce the recombinant protein. In particular, cell culture can be employed on an industrial scale by using vessels such as bioreactors. Cell culture in a bioreactor allows the cells to be placed in a medium that is ideal for the growth of the cell populations thus making it possible to obtain large quantities of these molecules of interest. The cells being cultured may be of all kinds of origin, for example animal, vegetable, bacterial or even yeast. For example, microalgae of the dinoflagellate type naturally contain anticancerous molecules or antalgic molecules. Also, the production of toxins is of major interest for performing toxicological studies.

Some of these species require a large addition of carbon dioxide or of oxygen in order to sustain or grow a population. Current cell culture methods meet this need by bubbling or by significant stirring using blades. However, these methods of adding gas are not suitable for culturing all types of species because they give to rise to a great deal of shear which destroys certain cells or creates significant cell stress, greatly reducing productivity. This is the case for example with certain microalgae.

Certain devices of the prior art allow gas to be added by means other than bubbling. Patent application US2015/0087049 A1 discloses a photobioreactor for the growth, at extremely high cell density, of axenic cultures of cyanobacteria and microalgae exposed to high intensities of light. A first hydrophobic membrane permeable to the gas is situated at the bottom of a reaction chamber to admit CO₂ into the cell suspension. A turbulent flow in the suspended cells is needed in order to achieve a high yield and this is obtained by stirring at at least 100 rpm and by creating a high shear rate, increasing the CO₂ absorption.

However, obtaining a turbulent flow using such a stirring method leads to a shear rate that is too high to sustain or allow the development of populations of numerous plant or animal species. In addition, such a shear rate for numerous species creates a state of cell stress that makes it impossible to achieve optimal conditions for the production of molecules of interest. Thus, such devices are limited in an industrial context and do not allow numerous industrially attractive species to be grown.

Methods of the prior art use other devices for cell culture. Specifically, U.S. Pat. No. 6,902,902 B2 describes a method for producing recombinant polypeptides by the use of mammalian cells which are transformed in a horizontal culture enclosure at low shear and in which oxygenation is achieved by means of an external gas exchange membrane in the flow.

However, such a horizontal enclosure is unable to produce enough turbulence in the liquid in which the cells are being cultured and this means that the gas exchange membrane rapidly becomes clogged with cells that become fixed thereto. Also, the liquid flow created by such a system prevents the distribution of the cell culture in the liquid from becoming uniform and is unable to optimize the surface areas for exchanges between the cells and the liquid or to improve the diffusion of light into the liquid. Such devices are therefore not viable on an industrial scale.

Application WO2017/149034 A1 discloses a bladeless device without any injection of air bubbles allowing a liquid to be stirred with a low shear rate within the liquid and a low risk of contamination of the liquid by comparison with a blade that has to be cleaned regularly, while at the same time dispensing with the need to use a complex device for injecting air bubbles. However, such systems are unable to add sufficient gas into the liquid for cell culture as the surface area for exchange between the liquid and the air is insufficient.

There is therefore a need for a cell culture system that allows a liquid to be mixed in order to obtain uniform distribution of the cells in the liquid while at the same time preventing the cells from blocking a large addition of gas, for example of oxygen or of carbon dioxide, but without reaching a shear rate that would prevent or reduce the production of molecules of interest.

The objective in particular is to obtain a production yield that is at least greater than that obtained by the earlier methods, e.g. by reducing the cost of obtaining these molecules and/or by increasing the quantity thereof produced.

### SUMMARY OF THE INVENTION

The invention relates to a cell culture system comprising: a first component that is cylindrical or frustoconical and of axis A and that has a first radius, the first component being open at the top and having at least one opening at the bottom, a membrane for diffusing a gas into a liquid by dissolution, the membrane being arranged at the lower part of the first component and positioned in such a way as to cover the at least one opening of said first component, the first component and the membrane being configured to form a container able to hold a liquid at a liquid head height H measured along the axis A, an enclosure able to hold a volume of gas, the enclosure being assembled with the container and arranged in such a way that the volume of gas can diffuse into the liquid via the membrane, and a drive member for driving the container in a rotational movement about the axis A, the container being inclined in such a way that the axis A forms a non-zero angle α that is less than or equal to 30° with respect to the vertical direction.

What is meant by a “vertical direction” is a direction parallel to the direction of gravity in an earth frame of reference.

What is meant by “height H measured along the axis A” is that the height H of the liquid is measured along the axis A and the intersection of the surface of the liquid with the axis A and/or along an axis A that is vertical. What is meant by “a container able to hold a liquid at a height H measured along the axis A” is that the container is tall enough in height that the liquid, when the container is inclined so as to form a non-zero angle α that is less than or equal to 30° with respect to the vertical direction, is held inside the container.

Advantageously, the container contains no blades. This absence of blades may apply to all the embodiments of the present invention.

As a preference, the first component is cylindrical.

Advantageously, the first component of said system is transparent to visible light. A transparent first component of said system allows the cell culture to receive light, this light being able to be used by certain cell cultures as a source of energy. A transparent system also allows the liquid in the first component to be monitored. If the cells being cultured are sufficiently clear, the transparent first component also allows the surface of the membrane in contact with the cell liquid to be monitored. What is meant by “transparent” is that the container has an optical transmission factor or total optical transmittance of at least 80%, preferably of at least 90%.

Advantageously, the first component is made from a polycarbonate, allowing all wavelengths of light to pass.

Also or as an alternative, the first component of said system is transparent to UV A and/or B and/or C radiation.

Advantageously, said system comprising the transparent first component comprises at least one light source arranged outside the first component and able to be directed toward the container. The at least one light source may comprise light emitting diodes, organic light emitting diodes, an incandescent bulb, or any other type of suitable light source. Such systems allow high powered illumination that is particularly well suited to the cell culture of species that require the addition of light.

Alternatively, the first component of said system is opaque to visible light. An opaque first component of said system allows the cell culture not to receive light, as such light may, for certain cell cultures, represent a source of stress which may trigger mechanisms that counter cell development and proliferation. What is meant by “opaque” is that the container has an optical transmission factor or a total optical transmittance of less than 2%, preferably of zero.

Advantageously, the first radius R1 of said first component is comprised between 5 cm and 100 cm.

Advantageously, the membrane is a porous membrane having pores of a diameter less than 75 nm or else is a dense membrane.

The membrane according to the invention for diffusing a gas into a liquid by dissolution is preferably impermeable to the liquid. What is meant by a “dense membrane” is a membrane that is free of porosities and through which the ions and molecules are transported through a solubilization-diffusion mechanism. What is meant by a “porous membrane” is a membrane that has porosities, the porosities being filled with gas in the case of hydrophobic membranes or with liquids in the case of hydrophilic membranes. In such instances, the gas is diffused through the gas or the liquid present in the pores because of the concentration gradient and dissolves into liquid on entering the liquid contained in the container.

In the alternative of a dense membrane, the dense membrane is made of polypropylene, of polytetrafluoroethylene, of polyvinylidene fluoride, of cellulose ester, of silicon or of a combination of at least two of these materials.

In the alternative of a porous membrane, the membrane is preferably a microporous or mesoporous membrane, preferably made of fluoropolymer, polyurethane, polyethylene, polypropylene or a combination of at least two of these materials. What is meant by a “mesoporous membrane” is a membrane of which the pore size is of the order of 2 to 50 nm. What is meant by a “microporous membrane” is a membrane of which the pore size is less than 2 nm.

Advantageously, the membranes of the invention are biologically inert and nontoxic with respect to the cells. Advantageously, the membranes of the invention may have undergone a surface treatment to make them particularly well suited to cell culture.

Advantageously, the container has a height H1 measured along the axis A that is suitable for holding the liquid at a height H measured along the axis A, even when it is inclined by the angle α, H1 being at least equal to H+R1×tan(α).

Advantageously, the system comprises a second component that is cylindrical or frustoconical, arranged inside the first component and having a second radius R2, said second radius being less than the first radius R1, the height H2 of the second component, measured along the axis A, being substantially identical to the height H1 of the first component, and the central axis of the second component being substantially identical to the axis A of the first component, the container able to hold the liquid being formed by the ring comprised between the first and second components. Such a system comprising a second component makes it possible to increase the surface area of the container that can come into contact with a liquid, also making it possible to increase the diffusion of light into the reactor. Advantageously, the second component of said system is transparent to visible light, this notably making it possible to increase the volume of liquid that is illuminated by the light.

As a preference, the second component is made from a material identical to the first component. As a preference, the second component is cylindrical.

Advantageously, regarding the systems comprising a cylindrical or frustoconical second component arranged inside the first component, the difference between the second radius R2 and the first radius R1 is less than 30 cm, preferably less than 20 cm. Such a system notably makes it possible to optimize the addition of light to the cell culture liquid by reducing the distance through which the light has to pass, thus also allowing better diffusion of light into the cell culture medium.

Advantageously, regarding systems comprising a cylindrical or frustoconical second component arranged inside the first component, said system comprises at least one light source arranged inside the second component and able to be directed toward the container. Such a system notably makes it possible to illuminate the cell culture liquid from inside the system and from outside the system and makes it possible to improve the addition of light to the cell liquid. Such systems allow particularly intensive illumination suited to the cell culture of species that require the addition of light.

The invention also relates to a cell culture system comprising: a first component that is cylindrical or frustoconical and of axis A, and that has a first radius R1, the first component having a height H1 measured along the axis A; a second component that is cylindrical or frustoconical, arranged inside the first component and having a second radius R2, the central axis of the second component being substantially equal to the axis A of the first component, said second radius R2 being less than the first radius R1, the second component having a height H2 substantially identical to the height H1 of the first component, the first component and the second component forming a ring-shaped container open at the top and able to hold the liquid at a height H measured along the axis A; and a drive member for driving the container in a rotational movement about the axis A, the container being inclined in such a way that the axis A forms a non-zero angle α that is less than or equal to 30° with respect to the vertical direction.

Such a system comprising a second component and forming a container in the shape of an open ring makes it possible to increase the surface area of the container that can be exposed to light and thus allows the cell culture in suspension in the liquid contained in the container to capture more light.

Advantageously, the first component and the second component are transparent to visible light and the system comprises at least a first light source arranged outside the first component and able to be directed toward the container, and at least one second light source arranged inside the second component and able to be directed toward the container.

Advantageously and regarding the systems that comprise a cylindrical or frustoconical second component arranged inside the first component, the aspect ratio H/R1 is comprised between 0.5 and 2. As a preference, the height H is chosen so as to cause resonance of an inertial mode of the rotating fluid, which is to say so that the height H satisfies the following equation (1):

$$3 {P}_{0} ( \frac{{3}^{\frac{1}{2}} π n {R}_{1}}{H} ) - {P}_{2} ( \frac{{3}^{\frac{1}{2}} π n {R}_{1}}{H} ) + 2 {P}_{1} ( \frac{{3}^{\frac{1}{2}} π n {R}_{1}}{H} ) \times {H}^{2} / ( {n}^{2} {π}^{2} {R}_{1} {R}_{2} ) = 0$$

where n is a non-zero natural whole number and the function Pₘ(x) is defined by:

$${P}_{m} ( x ) = {J}_{m} ( x ) \times {Y}_{m} ( \frac{x {R}_{2}}{{R}_{1}} ) - {J}_{m} ( \frac{x {R}_{2}}{{R}_{1}} ) \times {Y}_{m} ( x )$$

for m=0, 1, 2 and where Jₘ is the mth-order Bessel function of the first kind and Yₘ is the mth-order Bessel function of the second kind.

Advantageously, the aspect ratio H/R1 makes it possible to cause resonance of the first inertial mode when the aspect ratio H/R1 and the ratio of the radii R2/R1 are defined according to the curve of the graph in FIG. 5. As a preference, for a ratio of radii R2/R1=0.333, the aspect ratio H/R1 is comprised between 1.2 and 1.5, preferably between 1.3 and 1.4. Even more preferably, it is 1.35. When the aspect ratio between the height of the water H and the radius R1 is comprised between the above-indicated values, the number of rotations of the reactor according to the present invention is significantly reduced, as shown by the graph in FIG. 7.

Advantageously, the first radius R1 of said cylindrical or frustoconical first component is comprised between 5 cm and 100 cm.

Advantageously, the first component and the second component have a height H1 measured along the axis A that is suitable for holding the liquid at a height H measured along the axis, even when inclined by the angle α, H1 being at least equal to H+R1×tan(α).

As a preference, the second component is made from a material identical to the first component. As a preference, the first component and the second component are cylindrical (cylinders of revolution).

Advantageously, regarding systems comprising a cylindrical or frustoconical second component arranged inside the first component, the difference between the second radius R2 and the first radius R1 is less than 30 cm, preferably less than 20 cm. Such a system notably makes it possible to optimize the addition of light into the cell culture liquid by reducing the distance that the light has to pass through, also allowing better diffusion of light into the cell culture medium.

The invention also relates to a cell culture method using a system as described hereinabove, the method comprising a step of supplying a liquid containing a species to be cultured in the container up to a height H measured on the axis A and a step of rotating the container using the drive member.

Advantageously, the cell culture may be a culture of mammalian (human or non-human) cells such as mesenchymatous stem cells, embryonic stem cells, induced pluripotent stem cells, hematopoietic stem cells, T-lymphocytes (Car T-cells) etc. In the case of human embryonic stem cells, these are obtained only without destroying the embryo from which they are derived and are not of a nature to induce the process of development in a human being.

Advantageously, the cell culture may contain at least one species to be cultured from among microphytes. As a preference, the cell culture is a dinophyte culture. More preferably still, the cell culture contains at least one species to be cultured from among Alexandrium, Amphidinium, Azadinium, Dinophysis, Gambierdiscus toxicus, Gonyaulax, Gymnodinium mikimotoi, Karenia brevis, Lingulodinium, Ostreopsis, Prorocentrum and Protoceratium.

Advantageously, and regarding systems comprising a single cylindrical or frustoconical component, the aspect ratio H/R1 is comprised between 0.56 and 0.68, between 0.86 and 1.06 or between 1.79 and 2.19. Such aspect ratios in particular make it possible to generate resonance phenomena and allow effective mixing with a very low shear rate.

Advantageously, the rotational movement having an angular velocity of rotation Ω such that (Ω×R1²×α)/v, where v is the kinematic viscosity of the liquid, is greater than 1000. Such an angular velocity of rotation in particular makes it possible to provoke unstable resonance in the liquid and this further improves the mixing of the liquid.

The invention also relates to an assembly comprising a system as described hereinabove and a liquid containing a species to be cultured in the container of the system.

### BRIEF DESCRIPTION OF THE DRAWINGS

Further features, details and advantages of the invention will become apparent from reading the description which makes reference to the attached drawings which are given by way of example and which, respectively, depict:

FIG. 1 illustrates a view in section on a vertical plane of a first embodiment of a cell culture system comprising a container formed by a first component and a membrane.

FIG. 2 illustrates a view in section on a vertical plane of a second embodiment of a cell culture system comprising a container formed by a first component and a membrane and at least one light source.

FIG. 3 illustrates a view in section on a vertical plane of a third embodiment of a cell culture system comprising a container formed by a first component, a second component and a membrane, the system also comprising at least two light sources.

FIG. 4 illustrates a view from above of a cell culture system according to the third embodiment, comprising a container formed by a first component, a second component and a membrane, the system also comprising light sources.

FIG. 5 illustrates a graph indicating aspect ratios as a function of the ratios between the second radius and the first radius of a cell culture system comprising a container formed by a first component, a second component and a membrane according to the present invention.

FIG. 6 illustrates a view in section on a vertical plane of a fourth embodiment of a cell culture system comprising a container formed by a first component and a second component forming a ring, the system also comprising at least two light sources.

FIG. 7 corresponds to a graph indicating the mixing time Ωτ as a function of the ratio H/r where h is the height of water in the ring formed by the first component and the second component and measured on the axis A, and r is the ratio of the first radius R1 of the first component to the radius R2 of the second component.

### DETAILED DESCRIPTION

The systems and methods of the present invention are suitable for allowing cell culture by mixing the liquid in which the cells are to be found using turbulent flow through resonance phenomena. In particular, the shearing between the liquid containing the cell culture and the first component makes it possible to generate inertial waves thanks to the Coriolis force. These waves are reflected between the walls and the end wall of the container and create an overall movement of liquid in the container, the particles contained in the liquid following an elliptical path.

The systems and methods of the present invention notably make it possible to keep a cell culture in suspension with a uniform distribution of cells in the liquid while at the same time encouraging gas exchanges and preventing the cells from blocking off the supply of additional gas, for example of oxygen or of carbon dioxide. Also, the mixing of the cell culture that is brought about by the systems and methods of the present invention is able to avoid giving rise to a shear rate that might damage the cells, reduce their proliferation and cell development and that might prevent or reduce the production of molecules of interest by the one or more species being cultured.

The systems and the methods of the present invention thus make it possible to improve the yield by comparison with the present methods. Thus, the system amongst other things reduces the cost of obtaining molecules of interest by increasing the quantity thereof produced, for a cost equal to or lower than the costs of the earlier methods.

In certain embodiments of the invention, the systems and methods of the present invention comprise no blades. Blades used for mixing in cell culture containers give rise, within the cell culture liquid, to a shear rate that is too high for numerous species and prevent or reduce the sustaining or growth of a population of a species being cultured or the production of molecules of interest by a species being cultured.

In an embodiment suited to the culturing of stem cells, the radius R1 is advantageously comprised between 5 cm and 30 cm. In an embodiment suited to the culturing of microphytes, the radius R1 is advantageously comprised between 10 cm and 100 cm.

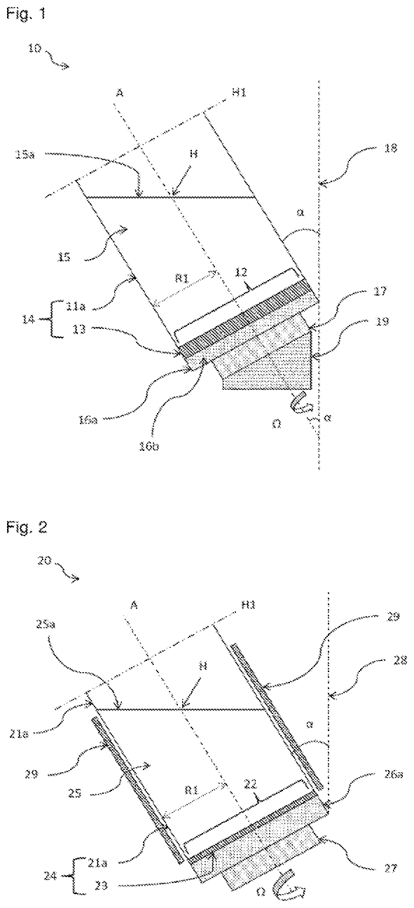

FIG. 1 illustrates a view in section of a vertical plane of a cell culture system 10 comprising a container formed by a first component 11a and a membrane 13. The first component 11a is cylindrical and has a first radius R1 and an axis A. The first component 11a is also open at the top and has at least one opening 12 at the bottom. In general, the top part of the first component extends as far as a plane perpendicular to the axis A and the bottom part of the first component extends as far as a plane perpendicular to the axis A, but other shapes are possible. Also, the first component has a height H1. In certain embodiments, the top part of the first component corresponds to the top end of the first component which does not come into contact with the liquid when the container is rotating, while the bottom part of the first component corresponds to that part of the first component that does come into contact with the liquid when the container is rotating. In such embodiments, at least one opening of the first component may be situated on the part of the first component that is parallel to the axis A, which is to say in the lateral wall of the bottom part of the first and/or of the second component. In such embodiments, the membrane covers this at least one opening and is therefore at least partially parallel to the axis A. In such embodiments, the membrane makes it possible to increase the surface area for exchange of gas and flow of gas from the enclosure toward the liquid by dissolution.

Also, the systems of the present invention may comprise a frustoconical first component of axis A and of first radius R1, where the first radius R1 corresponds to the largest radius of the frustoconical first component.

The cell culture system 10 comprises a membrane 13 for diffusing a gas into the liquid 15 by dissolution. The membrane 13 is arranged at the bottom part of the first component 11a and positioned in such a way as to cover the at least one opening 12 of the first component and form a container 14 able to hold the liquid 15 at a liquid head height H measured along the axis A. The height H1 of the container 14 of FIG. 1 is greater than H+R1×tan(α) because the free surface 15a of the liquid 15 intersects the axis A at the liquid head height H as defined previously and the container 14 needs to have sufficient height to hold the liquid 15 in the container 14 even when it is inclined by an angle α.

In general, the shape of the membrane offers a surface area that allows the gas to diffuse toward the liquid medium. As a preference, the membrane is circular and disk-shaped, making it possible to improve the surface area for diffusion between the gas and the liquid. However, other shapes are possible and the first component may at its bottom have an opening of a different shape and a membrane the shape of which corresponds to the shape of the opening so as to form the container. For example, the opening may be square in shape in a plane perpendicular to the axis A and circumscribed around the cylindrical first component. In such an example, the membrane covers at least the square-shaped opening.

In other embodiments, the first component may comprise several openings and the membrane may comprise several components covering the openings of said first component.

The cell culture system 10 comprises an enclosure 16a able to hold a volume of gas 16b, the enclosure being assembled with the container 14 and arranged in such a way that the volume of gas 16b can diffuse into the liquid 15 via the membrane 13. The enclosure 16a of FIG. 1 has the shape of a cylinder and is assembled with the container via the membrane 13. In general, the enclosure has a cylindrical or frustoconical shape because this is a shape that is suited to rotation, although other shapes are possible. The enclosure 16a of FIG. 1 is arranged beneath the membrane 13 but other arrangements are possible depending on the arrangement of the membrane and of the first component.

The enclosure 16a is able to hold a volume of gas which may be air, O₂, CO₂ or any other gas which when diffused into the liquid will allow the cell culture in the container to proliferate. For example, the gas may contain CO₂, for example at a partial pressure of the order of 1%. A person skilled in the art will be capable of adapting the composition of the gas in the enclosure 16a and the partial pressures of the various gases in such a way as to optimize the additions of the gases for cell culture by diffusion of the gas into the liquid via the membrane.

The cell culture system 10 comprises a drive member 17 for driving the container 14 in a rotational movement about the axis A. The drive member 17 is disk-shaped but may have absolutely any shape. The drive member 17 is arranged beneath the container 14 and beneath the enclosure 16a, although other arrangements are possible.

The container 14 of the cell culture system 10 is inclined so that the axis A forms an angle α of 30° with respect to the vertical direction 18, although the container of the present invention may be inclined by a non-zero angle α less than or equal to 30°. The system 10 of FIG. 1 comprises an inclination member 19 allowing the container 14 to be inclined by the desired angle. The inclination member 19 is situated beneath the drive member, although other arrangements are possible. The inclination member may also act as a support for the system 10 of the present invention.

FIG. 2 illustrates a view in section on a vertical plane of a cell culture system 20 comprising a container 24 formed by a first component 21a and a membrane 23 covering the opening 22 of the first component 21a, the system 20 also comprising at least one light source 29. A liquid 25 having a free surface 25a is arranged in the container 24. The light sources 29 of FIG. 2 are arranged around the first component 21a, e.g. at a distance from A that is greater than the radius R1. The membrane 23, the enclosure 26a able to hold a volume of gas, the drive member 27 and the vertical direction 28 are similar to the system 10 of FIG. 1. The system 20 of FIG. 2 comprises no inclination member, although an inclination member may be added to the system 20. Likewise, the first component has a height H1.

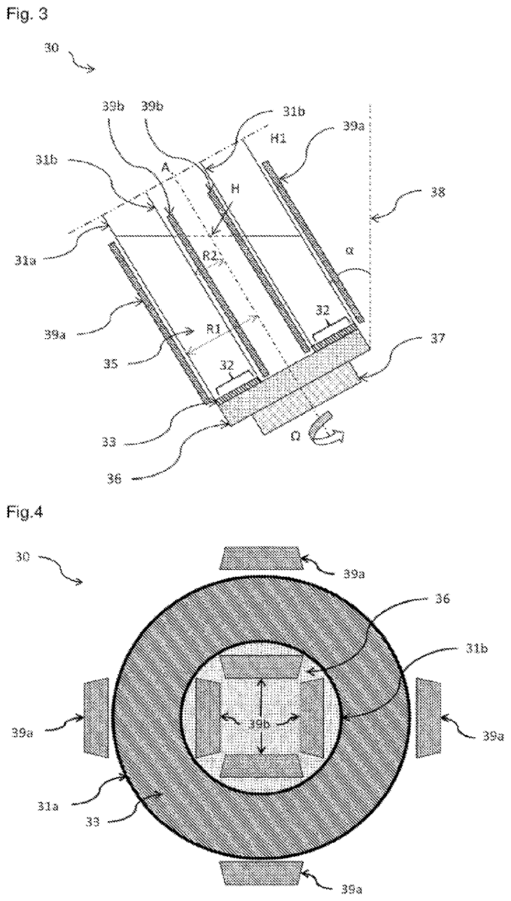

FIG. 3 illustrates a view in section on a vertical plane of a cell culture system 30 comprising a container formed by a first component 31a, a second component 31b and a membrane 33, the system 30 also comprising at least two light sources 39a and 39b. The light source 39a is arranged around the first component 31a, e.g. at a distance from A that is greater than the radius R1. The light source 39b is arranged inside the second component, e.g. at a distance from A that is smaller than the radius R2. Numerous forms of light source, for example in rings or in columns, may be employed in the systems of the present invention. Such a container may be referred to here as an annular container. The first component 31a is similar to the components 11a and 21a of FIGS. 1 and 2 respectively. The second component 31b is cylindrical and has a radius R2 less than the radius R1. In general, in the case of an annular container, the top part of the first component extends as far as a plane perpendicular to the axis A and the bottom part of the first component extends as far as a plane perpendicular to the axis A, although other shapes are possible.

Alternatively, in the case of an annular container, the systems of the present invention may comprise a frustoconical first component of axis A and of first radius R1, where the first radius R1 corresponds to the largest radius of the frustoconical first component, and a second component of second radius R2, where the second radius R2 corresponds to the largest radius of the frustoconical second component.

The height H1 of the first component 31a is substantially identical to the height

The container formed by the first component 31a, the second component 31b and the membrane 33 of the system 30 has the shape of a ring comprised between the first and second components. The opening 32, although viewed in section, is in the shape of a flat ring-shaped annulus corresponding to a disk of outside radius R1, of center A and of inside radius R2. The membrane 33 covers the opening 32 and therefore has a shape corresponding to this opening 32. Other shapes of opening and membrane are possible.

The enclosure 36 able to hold a volume of gas is arranged beneath the container and beneath the membrane 33. The enclosure 36 is in the shape of a disk rather than of an annular ring, but an enclosure in the shape of an annular ring is also possible. The drive member 37 and the vertical direction 38 are similar to the drive members and vertical directions of FIGS. 1 and 2.

FIG. 4 illustrates a view from above of a system 30 for the culture of cells in a liquid and comprising a container formed by a first component 31a, a second component 31b and a membrane 33, forming an annular container, the system also comprising light sources 39a and 39b.

The light sources 39a are arranged outside the annular container and the light sources 39b are arranged inside the annular container of FIG. 4. Although four external light sources 39a and four internal light sources 39b are described, the system of the present invention may comprise other light sources and/or light sources of different forms, for example in the form of an annular ring around the first component 31a and/or in the form of an annular ring inside the second component 31b. The liquid in the container is not depicted in FIG. 4, so that it is possible to see the gas enclosure 36 even though this is situated beneath the container of FIG. 4. The membrane 33 of the container is also visible, this in the case of FIG. 4 forming the bottom of the container. If a liquid were to be introduced into the system 30, this liquid would be introduced into the container, which is to say above the membrane 33, in the ring-shaped space between the first component 31a and the second component 31b.

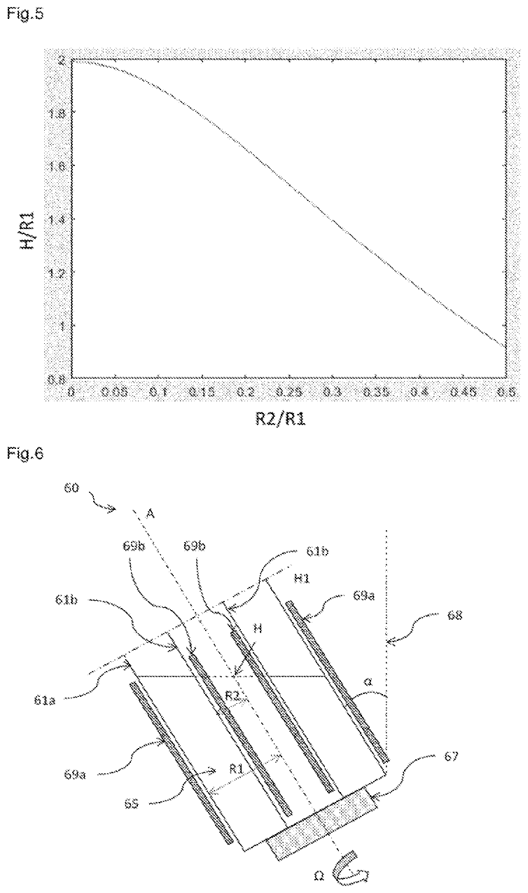

FIG. 5 illustrates a graph indicating aspect ratios as a function of ratios between the internal radius and the external radius of a cell culture system comprising a container formed by a first component, a second component and a membrane according to the present invention. In a system comprising an annular container, specific aspect ratios H/R1 in particular making it possible to generate resonance phenomena and making it possible to achieve effective mixing with a very low shear rate are determined from the ratio of the internal radius, corresponding to R2, to the external radius, corresponding to R1. This ratio corresponds to the greatest height H that satisfies equation (1) with n=1.

Using FIG. 5, and notably the curve of the graph in FIG. 5, several aspect ratios H/R1 can be determined as a function of the ratio of the radius R2 to the radius R1, R2/R1 being non-zero. A number of examples are set out in table 1 below:

TABLE 1

| Ratio of the radii R2/R1 | Aspect ratio H/R1 |
|---|---|
| 0.1 | 1.89 |
| 0.2 | 1.662 |
| 0.3 | 1.393 |
| 0.5 | 0.915 |
| 0.7 | 0.527 |

Examples of molecules of interest are set out as a function of the species of microphyte to be cultured in table 2 below:

TABLE 2

| Species | Molecules of interest |
|---|---|
| Alexandrium | Saxitoxin, cyclic imine, Tetrodotoxin, |
| | Goniodomin A |
| Amphidinium | Gonyautoxin, amphidinol, |
| | amphidinolide, Karlotoxin |
| Ostreopsis | Palytoxin |
| Protoceratium | Yessotoxin |
| Lingulodinium | Yessotoxin |
| Gonyaulax | Yessotoxin |
| Dinophysis | Pectenotoxin, Okadaic acid, |
| | Dinophysistoxin |
| Azadinium | Azaspiracid |
| Gymnodinium mikimotoi | Gymnocin |
| Prorocentrum | Okadaic acid, Dinophysistoxin |
| Gambierdiscus toxicus | Ciguatoxin, Maitotoxin, Gambierol, |
| | Gambieric acid |
| Karenia brevis | Brevetoxin |

In one particular embodiment, the membrane that allows a gas to diffuse into the liquid by dissolution is a laminated membrane with different types of fibers. Such a membrane may be employed in combination with all the embodiments of the invention.

In one particular embodiment, the system of the present invention comprises at least one means for determining at least one parameter of the liquid, a parameter being able to be selected from the temperature, the pH, the turbidity, the viscosity, the partial pressure of O₂, of CO₂ or some other gas. These determination means may be employed in combination with all the embodiments of the invention.

In one particular embodiment, the system comprises a means of agitating the gas in the enclosure able to hold a volume of gas. Agitating the gas in the enclosure allows the gas in the enclosure to be homogenized and makes it possible to reduce the pH fluctuations in the liquid resulting from the diffusion of the gas from the enclosure into the liquid. This can be achieved for example by recirculating the gas in the enclosure using an external pump connected to two ports of the enclosure. Such recirculation of the gas in the gas enclosure makes it possible to maintain a stream of gas toward the liquid by dissolution across the entirety of the membrane. Without recirculation, some parts of the membrane might not be continuously in contact with the gas that needs to be diffused into the liquid by dissolution.

In one particular embodiment, the system comprises a first component that is cylindrical and transparent and has a first radius of 50 cm, a second component that is cylindrical and concentric and transparent and has a second radius of 70 cm, LEDs outside the first component and LEDs inside on the second component. Such a system allows intense illumination particularly suited to the culturing of species that require the addition of light. For example, such a system allows intense illumination particularly well suited to the proliferation of dinoflagellates, for example with a determined wavelength and with an intensity of illumination comprised between 250 and 300 μmol·m⁻²·s⁻¹. For example, a 200 to 1000-liter system such as this containing a dinoflagellate cell culture allows a dinoflagellate duplication time of 0.95 days and a dinoflagellate productivity of 2.4 g·L⁻¹·day⁻¹, which is significantly higher than the productivity achieved using methods employing photobioreactors of the prior art for which a productivity of 0.16 g·L⁻¹·day⁻¹ and a duplication time of 5.87 days have been postulated (Fuentes-Grunewald, et al., 2012).

In one particular embodiment which may be combined with the other embodiments, the first component and/or the second component comprises at least one opening parallel to the axis A, which is to say in the lateral wall of the bottom part of the first and/or of the second component, this opening being covered by a membrane for diffusing a gas into the liquid by dissolution. According to such an embodiment, the enclosure able to hold a volume of gas is assembled with the first component and/or with the second component at least via those parts of the first and/or of the second component that are parallel to the axis A and that comprise the at least one opening, which is to say peripherally on the outside of the first component and/or on the inside of the second component. In such an embodiment, the system may also comprise means for recirculating the gas in the enclosure able to hold a volume of gas.

FIG. 6 illustrates a view in section on a vertical plane of a fourth embodiment of a cell culture system according to the present invention. The system comprises a cylindrical or frustoconical first component 61a of axis A, having a first radius R1, the first component 61a having a height H1 measured along the axis A; a cylindrical or frustoconical second component 61b, arranged inside the first component 61a and having a second radius R2, the central axis of the second component being substantially identical to the axis A of the first component 61a, said second radius R2 being less than the first radius R1, the second component 61b having a height H2 substantially identical to the height H1 of the first component 61a. The first component 61a and the second component 61b form a ring-shaped container open at the top and able to hold a liquid 65 at a height H measured along the axis A. The system also comprises a drive member 67 for driving the container in a rotational movement about the axis A, the container being inclined such that the axis A forms a non-zero angle α less than or equal to 30° with respect to the vertical direction 68. The first component 61a and the second component 61b are transparent to visible light and the system comprises at least one first light source 69a arranged outside the first component and able to be directed toward the container and at least one second light source 39b arranged inside the second component 31b and able to be directed toward the container.

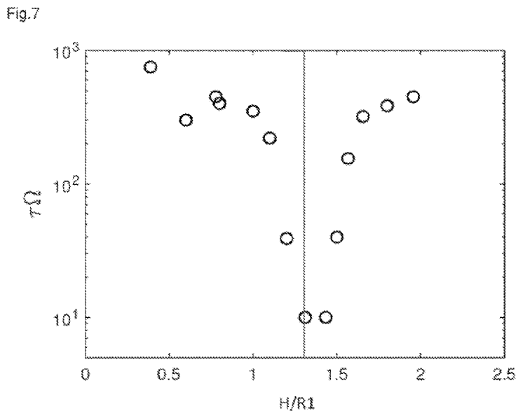

FIG. 7 corresponds to a graph indicating the mixing time T multiplied by the angular velocity Ω as a function of the ratio H/R1 where H is the height of the head of water in the ring formed by the first component and the second component and measured along the axis A for a radius ratio r=R2/R1 of the second component to the first component equal to 0.333. According to this graph, the mixing time OT varies as a function of the aspect ratio H/R1 so that the rotation time needed to obtain effective mixing is of the order of 200 to 400 rotations except when the aspect ratio H/R1 is comprised between 1.2 and 1.5 when the mixing time is significantly shorter, notably less than 100 or even of the order of 10. As a preference, the aspect ratio H/R1 is comprised between 1.3 and 1.4. Even more preferably, the aspect ratio H/R1 is around 1.35, which corresponds to the theoretical optimum aspect ratio indicated by a vertical line drawn as a solid line and derived from FIG. 5. Particularly in the case of this graph, the angle α is 3°, although other angles can be employed for the system of the present invention, which will nevertheless exhibit significantly shorter mixing times for obtaining effective mixing because of the aspect ratio H/R1 being comprised between 1.2 and 1.5.

The various embodiments set out in this description are nonlimiting and can be combined with one another. Further, the present invention is not restricted to the embodiments described hereinabove but extends to any embodiment that falls within the scope of the claims.

1. A cell culture system comprising:
a first component that is cylindrical or frustoconical and of axis A and that has a first radius R1, the first component being open at the top and having at least one opening at the bottom,
a membrane for diffusing a gas into a liquid by dissolution, the membrane being arranged at the lower part of the first component and positioned in such a way as to cover the at least one opening of said first component,
the first component and the membrane being configured to form a container able to hold a liquid at a liquid head height H measured along the axis A,
an enclosure able to hold a volume of gas, the enclosure being assembled with the container and arranged in such a way that the volume of gas can diffuse into the liquid via the membrane, and
a drive member for driving the container in a rotational movement about the axis A,
the container being inclined in such a way that the axis A forms a non-zero angle α that is less than or equal to 30° with respect to the vertical direction.

2. The system as claimed in claim 1, the first component being transparent to visible light.

3. The system as claimed in claim 2, comprising at least one light source arranged outside the first component and able to be directed toward the container.

4. The system as claimed in claim 1, the first component being opaque to visible light.

5. The system as claimed in claim 1, the first radius R1 being comprised between 5 cm and 100 cm.

6. The system as claimed in claim 1, the membrane being a porous membrane having pores of a diameter less than 75 nm or else being a dense membrane.

7. The system as claimed in claim 1, the container having a height H1 measured along the axis A that is suitable for holding the liquid at a height H measured along the axis A, even when it is inclined by the angle α, H1 being at least equal to H+R1×tan.

8. The system as claimed in claim 1, comprising a second component that is cylindrical or frustoconical, arranged inside the first component and having a second radius R2, said second radius being less than the first radius R1, the height H2 of the second component being substantially identical to the height H1 of the first component, and the central axis of the second component being substantially identical to the axis A of the first component, the container able to hold the liquid being formed by the ring comprised between the first and second components.

9. The system as claimed in claim 8, the difference between the second radius R2 and the first radius R1 being less than 30 cm, preferably less than 20 cm.

10. The system as claimed in claim 8, said system comprising at least one light source arranged inside the second component and able to be directed toward the container.

11. A cell culture system comprising:
a first component that is cylindrical or frustoconical and of axis A, and that has a first radius R1, the first component having a height H1 measured along the axis A;
a second component that is cylindrical or frustoconical, arranged inside the first component and having a second radius R2, the central axis of the second component being substantially identical to the axis A of the first component, said second radius R2 being less than the first radius R1, the second component having a height H2 substantially identical to the height H1 of the first component;
the first component and the second component forming a ring-shaped container open at the top and able to hold a liquid at a height H measured along the axis A; and
a drive member for driving the container in a rotational movement about the axis A,
the container being inclined in such a way that the axis A forms a non-zero angle α that is less than or equal to 300 with respect to the vertical direction.

12. The cell culture system as claimed in claim 11, the first component and the second component being transparent to visible light and the system comprising at least one first light source arranged outside the first component and able to be directed toward the container, and at least one second light source arranged inside the second component and able to be directed toward the container.

13. The cell culture system as claimed in claim 11, the height H being chosen so as to satisfy the following equation (1):
$$3 {P}_{0} ( \frac{{3}^{\frac{1}{2}} π n {R}_{1}}{H} ) - {P}_{2} ( \frac{{3}^{\frac{1}{2}} π n {R}_{1}}{H} ) + 2 {P}_{1} ( \frac{{3}^{\frac{1}{2}} π n {R}_{1}}{H} ) \times {H}^{2} / ( {n}^{2} {π}^{2} {R}_{1} {R}_{2} ) = 0$$
where n is a non-zero natural whole number and the function Pₘ is defined by:
$${P}_{m} ( x ) = {J}_{m} ( x ) \times {Y}_{m} ( \frac{x {R}_{2}}{{R}_{1}} ) - {J}_{m} ( \frac{x {R}_{2}}{{R}_{1}} ) \times {Y}_{m} ( x )$$
for m=0, 1, 2 and where Jₘ is the mth-order Bessel function of the first kind and Yₘ is the mth-order Bessel function of the second kind.

14. The cell culture system as claimed in claim 13, the aspect ratio H/R1 and the ratio of radii R2/R1 being defined according to the curve in the graph of FIG. 5, R2/R1 being non-zero.

15. The cell culture system as claimed in claim 11, the ratio of radii R2/R1 being equal to 0.333 and the aspect ratio H/R1 being comprised between 1.2 and 1.5, preferably being comprised between 1.3 and 1.4, and even more preferably being 1.35.

16. A cell culture method using a system as claimed in claim 1, the method comprising:
a step of supplying a liquid containing a species to be cultured in the container up to a height H measured on the axis A,
a step of rotating the container using the drive member.

17. A cell culture method using a system as claimed in claim 1, the method comprising:
a step of supplying a liquid containing a species to be cultured in the container up to a height H measured on the axis A, and
a step of rotating the container using the drive member,
the aspect ratio H/R1 being comprised between 0.56 and 0.68, between 0.86 and 1.06 or between 1.79 and 2.19.

18. The method as claimed in claim 16, the rotational movement having an angular velocity of rotation Ω such that (Ω×R1²×α)/ν, where ν is the kinematic viscosity of the liquid, is greater than 1000.

19. A cell culture method using a system as claimed in claim 11, the method comprising:
a step of supplying a liquid containing a species to be cultured in the container up to a height H measured on the axis A, and
a step of rotating the container using the drive member,
the aspect ratio H/(R1) being comprised between 0.5 and 2.

20. An assembly comprising a system as claimed in claim 1 and a liquid containing a species to be cultured in the container of the system.
